(19) United States
(12) Patent Application Publication
(10) Pub. No.: US 2025/0176850 A1
(43) Pub. Date: Jun. 5, 2025

(54) FLUID CONYEVING DEVICE FOR INTRODUCING FLUID INTO AND/OR REMOVING FLUID FROM AN ESOPHAGEAL BALLOON CATHETER

(71) Applicant: Hamilton Medical AG, Bonaduz (CH)

(72) Inventors: David Schwenninger, Felsberg (CH); Kevin Bitterli, Paspels (CH); Peter Steinbacher, Vättis (CH)

(73) Assignee: Hamilton Medical AG, Bonaduz (CH)

(21) Appl. No.: 18/841,005

(22) PCT Filed: Jan. 30, 2023
(86) PCT No.: PCT/EP2023/052125

§ 371 Date: Aug. 23, 2024

(30) Foreign Application Priority Data
Feb. 23, 2022 (DE) 10 2022 104 339.8

(51) Int. Cl. A61B 5/03; A61B 5/00; A61M 16/00; A61M 16/04; A61M 25/00; A61M 25/10

(52) U.S. Cl. CPC A61B 5/037; A61B 5/4836; A61B 5/6853; A61M 16/0057; A61M 16/04; A61M 2025/0001; A61M 25/10181; A61M 25/10185

(57) ABSTRACT

A fluid conveying device (65) for introducing fluid into and/or removing fluid from an esophageal catheter (48) which is insertable into an esophagus (34) and has a balloon probe (46) for determining an esophageal balloon pressure, comprising: a measuring fluid port (90), which is configured to be coupled to a distal end (48b) of the esophageal catheter (48) such that, in the coupled state, fluid is introducable into the esophageal catheter (48) and/or removable from the esophageal catheter (48) via the measuring fluid port (90); a pump device (100) having at least one fluid pump (102), wherein the pump device (100) is configured to convey fluid in at least a first conveying direction, and a switching valve (106) which is associated with the at least one fluid pump (102) and which is switchable at least between a first position and a second position. The at least one fluid pump (102) is in the form of a micropump or diaphragm pump.

### BACKGROUND OF THE INVENTION

The present invention relates to a fluid conveying device for introducing fluid into and/or removing fluid, in particular measuring fluid, into or from an esophageal balloon catheter which is insertable into an esophagus and has a balloon probe. The invention also relates to a device for determining an esophageal balloon pressure with a fluid conveying device according to the invention and to a ventilation device comprising such a device for determining an esophageal balloon pressure.

Catheters with balloon probe for determining an esophageal balloon pressure (“esophageal balloon catheter”) are used, in particular, in mechanical ventilation for determining the transpulmonary pressure in the chest of a patient.

### SUMMARY OF THE INVENTION

In today's common forms of mechanical ventilation, the patient is supplied with breathing gas at a positive pressure. Therefore, during ventilation, the airway pressure or alveolar pressure is greater than the pressure in the pleural space or gap surrounding the pulmonary alveoli, at least during the inspiration phase. During the expiration phase, there is no pressurization of the airway by the ventilation device, with the result that the lung tissue relaxes and the airway pressure or alveolar pressure decreases. Under certain circumstances, this kind of positive pressure ventilation may cause the pressure conditions in the airway or in the alveoli to become so unfavorable at the end of the expiration phase that parts of the alveoli collapse. The collapsed part of the lung volume then must first be unfolded again in the subsequent breathing cycle. The functional residual capacity of the lungs is severely impaired, so that oxygen saturation decreases, and the lung tissue also suffers permanent damage.

To prevent collapse of alveoli at the end of the expiration phase, a so-called positive end-expiratory pressure, commonly briefly referred to as PEEP, is usually set during positive pressure mechanical ventilation. With this measure, an improvement in oxygen saturation may be achieved in many cases.

When ventilating with PEEP, the ventilation device applies a predetermined positive pressure, the PEEP, to the airway permanently—i.e. both during the inspiration phase and during the expiration phase. The PEEP is therefore still present after the end of the expiration phase.

Ideally, the PEEP should be set sufficiently high so that during the expiration phase the alveolar pressure is not, or at least only to such an extent, below the pressure in the pleural gap that the alveolar tissue does not collapse under the effect of the pressure in the pleural gap. In other words, the PEEP is to prevent the transpulmonary pressure—which is the pressure difference between alveolar pressure and pressure in the pleural gap—from becoming lower than zero or from falling below a lower negative limit value at which parts of the alveoli begin to collapse.

On the other hand, a too high value of the PEEP may have a negative effect, especially during the inspiration phase. This is because the lung tissue can become overstretched at very high airway pressures during the inspiration phase. Numerous studies also indicate that a high value of the PEEP can impede the return flow of venous blood to the heart with corresponding negative effects on the cardiovascular system.

In itself, the PEEP should be matched to the respective prevailing transpulmonary pressure. However, the transpulmonary pressure in a ventilated patient is not amenable to simple determination. To get by, one therefore measures the pressure in a balloon probe placed in the esophagus of a ventilated patient by means of an esophageal balloon catheter. With suitable positioning and configuration of the balloon, the pressure measured in the balloon can be used to approximately determine the pressure in the pleural gap.

WO 2014/037175 A1 describes the automated setting of a pressure specified by a ventilation device, in particular the positive end-expiratory pressure (PEEP) and the maximum airway pressure, on the basis of the esophageal balloon pressure, which is regarded as an indicator of the transpulmonary pressure, i.e. the pressure difference between alveolar pressure and pressure in the pleural gap.

In practice, the relationship between the pressure measured in a balloon catheter inserted into the esophagus and the pressure in the pleural gap may change during ventilation. The causes for this can be manifold and generally cannot be determined in detail.

Mojoli et al, Crit. Care (2016) 20:98 and Holz et al., Respir. Care (2018) 63 (2): 177-186, recommend procedures for calibrating an esophageal balloon catheter with balloon probe. The aim of the calibration is to achieve optimal filling of the balloon catheter with air, in which the balloon catheter is as sensitive as possible to changes in the pressure in the pleural gap acting on the esophagus and reflects the pressure in the pleural gap as well as possible.

The measuring procedures described by Mojolo et al. for calibration, however, are complex and sensitive, so that in practical application merely an optimal filling for the balloon probe can be determined and preset before beginning ventilation. This optimal filling is then maintained during ventilation and is not changed any more.

There is therefore a need to enable a simpler and more accurate determination of the esophageal balloon pressure using an esophageal balloon catheter, in particular as a basis for determining a pressure in the pleural gap to determine the transpulmonary pressure during mechanical ventilation. In addition, largely automatic calibration of a system for determining an esophageal balloon pressure as a surrogate for the pressure in the pleural gap is to be rendered possible.

For the operation and calibration of an esophageal balloon catheter during ventilation, in addition to sufficiently accurate pressure measurement, the control of the amount of measuring fluid in the esophageal balloon catheter must be taken into account as well.

EP 3 197 348 B1 suggests to use a syringe pump known in the medical field as a fluid conveying device for filling and emptying an esophageal balloon catheter.

The present invention solves the problem of providing an improved fluid conveying device for filling and emptying an esophageal balloon catheter, which operates reliably and can be realized at low cost.

The invention comprises a fluid conveying device for introducing fluid into and/or removing fluid from a catheter with a balloon probe (“esophageal balloon catheter”), which is insertable into an esophagus, for determining an esophageal balloon pressure, wherein the fluid conveying device comprises: a measuring fluid port for connection to an esophageal balloon catheter, a pump device with at least one fluid pump which is configured to convey fluid in at least a first conveying direction, and a switching valve associated with the fluid pump.

The esophageal balloon catheter will be referred to simply as “catheter” in the following.

The measuring fluid port is configured to be coupled to a distal end of a catheter such that, in the coupled state, fluid is introducible into the catheter and/or removable from the catheter via the measuring fluid port. The switching valve is switchable at least between a first position and a second position. The fluid pump may be designed as a diaphragm pump or as a micropump, in particular as a piezo pump.

Micropumps and diaphragm pumps work quickly and precisely, especially when there are frequent changes between the introduction of fluid into the catheter and the removal of fluid from the catheter. The number of components installed both in micropumps and in diaphragm pumps is low. In particular, both types of fluid pumps contain only few components that are susceptible to wear, making fluid pumps of both construction types durable and cost-effective to manufacture. Micropumps allow for a particularly compact construction, so that they can also be used where space is at a premium. Diaphragm pumps and micropumps according to the invention are very quiet in operation. Their design is also less complex than the design of syringe pumps, and in particular they comprise fewer moving elements, making them more reliable in operation and less susceptible to maintenance than syringe pumps. As a rule, higher mass flows can be achieved with diaphragm pumps than with micropumps.

In the following, the term “conveying direction” refers to the direction of flow of the measuring fluid conveyed by the fluid pump in relation to the catheter.

The “coonveying direction” thus defined must be distinguished from the direction of flow of the measuring fluid through the fluid pump itself. Since the direction of flow of the measuring fluid through the fluid pump itself cannot be reversed, i.e. the direction of flow within the fluid pump is fixed, the fluid pump always conveys the fluid from an inlet of the fluid pump to an outlet of the fluid pump. The conveying direction, i.e. the direction of flow of the measuring fluid in relation to the catheter, therefore results from the fact that the measuring fluid port is connected, in particular by the switching valve, either to the inlet or to the outlet of the fluid pump.

When the outlet of the fluid pump is connected to the measuring fluid port, the first flow direction of the fluid pump corresponds to the introduction of fluid into the catheter. When the inlet of the fluid pump is connected to the measuring fluid port, the first flow direction of the fluid pump corresponds to the removal of fluid from the catheter.

In the following, the terms “inlet” and “outlet” of the fluid pump always refer to the flow direction of fluid through the respective fluid pump itself and not to the conveying direction which is switchable by the switching valve.

A fluid conveying device according to the invention may comprise a first fluid pump and a second fluid pump. The first fluid pump may be configured and arranged for conveying fluid in a first conveying direction and the second fluid pump may be configured and arranged for conveying fluid in a second conveying direction.

In particular, the first conveying direction may be opposite to the second conveying direction. Thus, the first conveying direction may correspond to the introduction of fluid into the catheter, and the second conveying direction may correspond to the removal of fluid from the catheter, or vice versa.

In the fluid conveying device with at least two fluid pumps, the introduction of fluid into the catheter and the removal of fluid from the catheter are performed by two different fluid pumps. For example, the introduction of fluid into the catheter is performed by the first fluid pump and the removal of fluid from the catheter is performed by the second fluid pump, or vice versa. With such a design, each fluid pump can be optimized for its respective conveying direction. Only a single switching valve is required to switch the conveying direction of the fluid conveying device between introduction and removal of fluid.

The first and second fluid pumps may be arranged and connected in parallel to each other. “Connected in parallel” in this context means that, depending on the position of the switching valve, either an outlet of the first fluid pump is connected to the measuring fluid port or an inlet of the second fluid pump is connected to the measuring fluid port. The switching valve is thus configured such that it connects the measuring fluid port selectively either to the outlet of the first fluid pump or to the inlet of the second fluid pump.

In particular, the switching valve is arranged between the outlet of the first fluid pump and the measuring fluid port and between the inlet of the second fluid pump and the measuring fluid port.

The second fluid pump can also be designed as a micropump or as a diaphragm pump. The first fluid pump and the second fluid pump can both be designed as micropumps or both as diaphragm pumps, so that both fluid pumps have similar or even substantially identical operating characteristics. In particular, the first fluid pump and the second fluid pump can be of identical design.

If different operating characteristics, in particular pressures and/or conveying capacities, are desired for the introduction of fluid into the catheter and for the removal of fluid from the catheter, the first fluid pump and the second fluid pump can also be designed differently so that they have different operating characteristics.

In particular, the first and/or the second fluid pump may be configured such that they are capable of generating a maximum fluid pressure in a range from 50 hPa to 150 hPa, in particular a maximum fluid pressure in a range from 80 hPa to 100 hPa, and provide a flow rate in a range from 60 ml/min to 200 ml/min, in particular a flow rate in a range from 70 ml/min to 100 ml/min.

Fluid pumps designed as piezo pumps can be operated at a frequency in a range between 100 Hz and 1500 Hz, in particular a frequency in a range between 800 Hz and 1000 Hz, when operating in a fluid conveying device according to the invention. In order to achieve the desired conveying capacity, the fluid pumps can be operated in particular at a higher frequency than specified by the respective manufacturer's specification.

The switching valve may be switchable between at least one first and at least one second open position. When the switching valve is switched to the first open position, the outlet of the first fluid pump may be fluidly connected to the measuring fluid port and the inlet of the second fluid pump may be fluidly disconnected from the measuring fluid port. When the switching valve is switched to the second open position, the inlet of the second fluid pump may be fluidly connected to the measuring fluid port and the outlet of the first fluid pump may be fluidly disconnected from the measuring fluid port.

“Fluidly connected” in this context means that there is a fluid connection between the measuring fluid port and the first or second fluid pump, so that fluid may be conveyed from the first or second fluid pump to the measuring fluid port. “Fluidly disconnected” in this context means that there is no fluid connection between the measuring fluid port and the first or second fluid pump, so that no fluid may be conveyed from the first or second fluid pump to the measuring fluid port.

In a fluid conveying device configured in this way, the first fluid pump conveys fluid into the catheter (introduction of fluid) when the switching valve is in the first open position, and the second fluid pump conveys fluid from the catheter (removal of fluid) when the switching valve is in the second open position.

The switching valve may comprise a first release valve associated with the first fluid pump and a second release valve associated with the second fluid pump. The first release valve may be switchable at least between an open position, in which the outlet of the first fluid pump is fluidly connected to the measuring fluid port, and a closed position, in which the outlet of the first fluid pump is fluidly disconnected from the measuring fluid port. The second release valve may be switchable at least between an open position, in which the inlet of the second fluid pump is fluidly connected to the measuring fluid port, and a closed position, in which the inlet of the second fluid pump is fluidly disconnected from the measuring fluid port.

In particular, the first and second release valves may be arranged in parallel to each other so that fluid may flow through the switching valve when at least one of the two release valves is in its open position.

In this way, the function of the switching valve may be realized in cost-effective and reliable manner by two release valves, which are simple valves that each have only one open position and one closed position.

The switching valve and, in particular, the release valves may be configured as mechanically, electrically, hydraulically and/or pneumatically controllable valves.

The first and second release valves may be coordinated or synchronized with each other such that they can only be switched over together, so that when the first release valve is in the open position, the second release valve is in the closed position, and when the first release valve is in the closed position, the second release valve is in the open position. This ensures that a respective one of the two release valves is open and closed at all times, thus reliably preventing a malfunction of the switching valve in which both release valves are open or closed at the same time.

The release valves may be mechanically, electrically, hydraulically and/or pneumatically coordinated or synchronized with each other.

The switching valve may also be configured such that it fluidly connects the measuring fluid port selectively either to an inlet of the (first) fluid pump or to an outlet of the (first) fluid pump. With a switching valve configured in this way, a fluid conveying device may be created which has only a single fluid pump and which may be switched between two opposite conveying directions, in particular a first conveying direction for introducing fluid into a catheter and a second conveying direction for removing fluid from a catheter.

A second fluid pump may be dispensed with in such a fluid conveying device, which means that the costs of the fluid conveying device can be kept low.

Dispensing with a second fluid pump is particularly advantageous if a diaphragm pump is used as fluid pump, as diaphragm pumps are generally more expensive than micropumps and require a larger installation space.

A fluid conveying device comprising a switching valve that is configured such that it fluidly connects the measuring fluid port selectively either to an inlet of a fluid pump or to an outlet of the fluid pump may also be equipped with at least one additional fluid pump.

For example, a second fluid pump may be connected in series with the first fluid pump in order to increase the maximum achievable output pressure of the fluid. The maximum achievable fluid flow may be increased by connecting at least two fluid pumps in parallel.

Such a series or parallel connection works with diaphragm pumps and with micropumps/piezo fluid pumps. A parallel or series connection of at least two fluid pumps is particularly advantageous when micropumps/piezo pumps are used as fluid pumps, as these often have a lower conveying capacity than diaphragm pumps, in particular, a conveying capacity that is lower than the conveying capacity desired for introducing and/or removing fluid into or from a catheter.

The switching valve may comprise a combination of several switching valves. In particular, the switching valve may comprise a first conveying direction switching valve and a second conveying direction switching valve, wherein the first conveying direction switching valve and the second conveying direction switching valve are arranged in series with each other with respect to the conveying direction of fluid through the fluid pump. “Arranged in series with each other” in this context means that the first and second conveying direction switching valves are arranged one behind the other in the conveying direction of the measuring fluid, and that no fluid could be conveyed through the fluid pump if (hypothetically) at least one of the two conveying direction switching valves were in a blocking position in which it blocks the flow of fluid therethrough.

The first conveying direction switching valve may be located downstream of the fluid pump with respect to the direction of flow of fluid through the fluid pump. The second conveying direction switching valve may be located upstream of the fluid pump with respect to the direction of flow of fluid through the fluid pump.

The conveying direction switching valve may be associated with or located adjacent to the outlet of the fluid pump and fluidly connected to the outlet of the fluid pump. The second conveying direction switching valve accordingly may be associated with or located adjacent to the inlet of the fluid pump and fluidly connected to the inlet of the fluid pump.

The first conveying direction switching valve may be switchable between at least a first open position and at least a second open position, wherein in the first open position the outlet of the fluid pump is fluidly connected to the measuring fluid port and in the second open position the outlet of the fluid pump is fluidly disconnected from the measuring fluid port.

Similarly, the second conveying direction switching valve may be switchable between at least a first and a second open position, wherein in the first open position the inlet of the fluid pump is fluidly disconnected from the measuring fluid port and in the second open position the inlet of the fluid pump is fluidly connected to the measuring fluid port.

In this way, a fluid conveying device may be realized with a single fluid pump, which makes it possible to switch the fluid conveying direction of the fluid conveying device in a targeted manner. The costs and installation space for a second fluid pump may thus be saved.

The first and second conveying direction switching valves may be coupled to each other such that they can only be switched in coordinated or synchronized manner with each other, in particular such that when the first conveying direction switching valve is in the first open position, the second conveying direction switching valve is also in the first open position. This may correspond to an operating state in which the operation of the fluid pump leads to the introduction of fluid into the catheter.

The first and second conveying direction switching valves may be coupled to each other such that they can only be switched in coordinated or synchronized manner with each other, in particular such that when the first conveying direction switching valve is in the second open position, the second conveying direction switching valve is also in the second open position. This may correspond to an operating state in which the operation of the fluid pump leads to the removal of fluid from the catheter.

By coupling the conveying direction switching valves in this way, a combination of open positions of the two conveying direction switching valves, which does not correspond to a desired operating state of the fluid conveying device, can be reliably avoided.

The first and second conveying direction switching valves may be actuated mechanically, electrically, pneumatically and/or hydraulically.

In particular, the first and second conveying direction switching valves may be coupled to each other mechanically, electrically, pneumatically and/or hydraulically.

By selectively, possibly temporarily, removing the coupling between the conveying direction switching valves, special operating states may be set as required in which the two conveying direction switching valves are in different open positions and in which fluid is neither conveyed into the catheter nor removed from the catheter.

Such special operating states may be set, for example, to bring the conveying capacity of the fluid pump to a defined state, e.g. after starting up the fluid pump, before fluid is conveyed from the fluid pump into the catheter, and/or to convey fluid in a circuit, e.g. to flush the components of the fluid conveying device with fluid.

The fluid conveying device may also have a valve with a blocking function that enables the pump device to be fluidly disconnected or separated from the measuring fluid port so that no fluid can flow between the pump device and the measuring fluid port.

In particular, the fluid conveying device may comprise a blocking valve which is arranged downstream of the fluid pump and which has at least one blocking position in which the pump device is disconnected from the measuring fluid port.

The blocking valve may be a valve that is separate from the switching valve. Such a separate blocking valve may be arranged downstream in series with the switching valve; in particular, it may be arranged between the switching valve and the measuring fluid port in order to be able to fluidly disconnect the measuring fluid port, and in particular a catheter connected to the measuring fluid port, from the fluid pump.

There may also be used a switching valve which has a blocking position in which the pump device is fluidly disconnected from the measuring fluid port. In this case, a separate switching valve may be dispensed with.

Each fluid pump may have associated therewith a device for forming a flow resistance, in particular a constriction of the flow cross-section. In particular, such a device may be configured such that it reduces the fluid flow achievable at the maximum pumping capacity of the respective fluid pump to a predetermined value due to its flow resistance. Damage to a catheter connected to the fluid conveying device and/or falsification of measurement results due to excessive fluid flow can thus be avoided. The use of a device for forming a flow resistance is particularly advantageous if at least one diaphragm pump is used as fluid pump, since diaphragm pumps generally have a higher nominal conveying capacity than micropumps/piezo fluid pumps. In particular, diaphragm pumps often have a nominal conveying capacity that is greater than is required for filling and emptying catheters in the context described here.

With the aid of a device for forming a flow resistance, the amount of fluid conveyed may be reduced to a flow rate suitable for the catheter without having to change the nominal conveying capacity of the fluid pump. The fluid pump may thus continue to be operated in its optimum working range.

The device for forming a flow resistance may be arranged in series with the respective fluid pump in the direction of flow. The device for forming a flow resistance may in particular be arranged upstream at the inlet and/or downstream at the outlet of the respective fluid pump.

If the fluid conveying device has more than one fluid pump, a common device for forming a flow resistance can be provided, which is associated with several, in particular all, fluid pumps. There may also be provided a separate device for forming a flow resistance for each fluid pump.

Such devices may be arranged, for example, downstream of the outlet of the first fluid pump and upstream of the inlet of the second fluid pump and/or upstream of the inlet of the first fluid pump and downstream of the outlet of the second fluid pump.

A first device for forming a flow resistance may be arranged in particular between the outlet of the first fluid pump and the measuring fluid port. A second device for forming a flow resistance may be arranged in particular between the inlet of the second fluid pump and the measuring fluid port. In this case, the first and second devices for forming a flow resistance are parallel to each other.

A common device for forming a flow resistance may be arranged such that, depending on the position of the switching valve, it is arranged between the outlet of the first fluid pump and the measuring fluid port or between the inlet of the second fluid pump and the measuring fluid port.

Instead of or in addition to at least one device for forming a flow resistance, there may also be provided at least one bypass which passes part of the fluid conveyed by the at least one fluid pump past the catheter in order to reduce the amount of fluid conveyed into the catheter and/or the fluid pressure in the catheter.

A fluid conveying device according to the invention may further comprise a flow sensor configured to detect the fluid flow conveyed into or out of the catheter by the first and/or the second fluid pump.

In particular, the flow sensor may be configured to detect a mass flow of the fluid conveyed into or out of the catheter or to detect a volume flow of the fluid conveyed into or out of the catheter. To this end, the flow sensor may be arranged in particular between the measuring fluid port and the at least one fluid pump.

A fluid conveying device according to the invention may also comprise a pressure sensor which is configured to measure a fluid pressure. The pressure sensor in particular may be configured to measure the fluid pressure in a region of a fluid conveying line between the measuring fluid port and the first or the second fluid pump.

The invention also comprises a device for detecting an esophageal balloon pressure on the basis of a catheter with a balloon probe (“esophageal balloon catheter”) which is insertable into an esophagus, the device comprising a fluid conveying device according to the invention.

The invention also comprises a ventilating device which comprises such a device for detecting an esophageal balloon pressure with a fluid conveying device according to the invention. The ventilating device in particular may be configured for the automatic determination of a transpulmonary pressure on the basis of the measured esophageal balloon pressure.

### BRIEF DESCRIPTION OF THE DRAWINGS

In the following, the exemplary embodiments described hereinbefore and further embodiments of the invention will be described in more detail with reference to the attached figures.

FIG. 1 shows a schematic view of an esophageal balloon catheter that is insertable into the esophagus of a patient, and a system for characterizing the esophageal balloon catheter, which comprises a fluid conveying device according to the invention.

FIG. 2 shows a schematic view of a fluid conveying device according to a first exemplary embodiment of the invention.

FIG. 3 shows a schematic view of a fluid conveying device according to a second exemplary embodiment of the invention.

FIG. 4A shows a fluid conveying device according to a third exemplary embodiment of the invention in a first operating state.

FIG. 4B shows a fluid conveying device according to a third exemplary embodiment of the invention in a second operating state.

FIG. 5 shows a schematic representation of the essential elements of a ventilation device according to the invention with a system for characterizing an esophageal balloon catheter, which comprises a fluid conveying device according to the invention.

### DETAILED DESCRIPTION

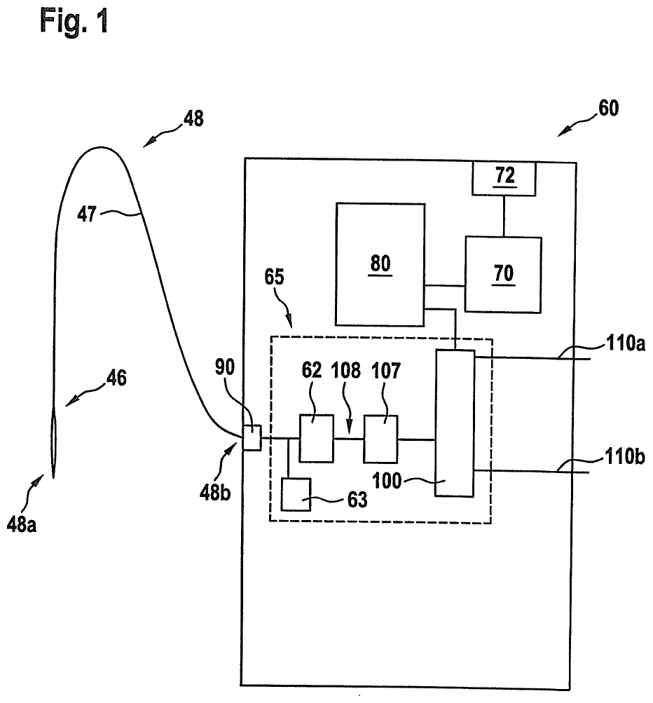

FIG. 1 shows a simplified schematic representation of an esophageal balloon catheter 48, hereinafter referred to as “catheter 48”, and a device 60 for detecting an esophageal balloon pressure.

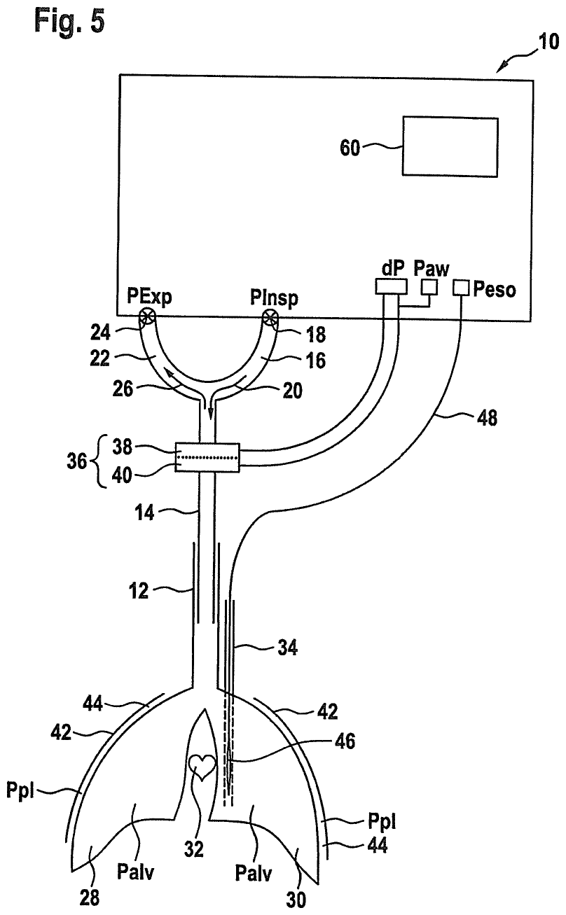

The catheter 48 comprises a catheter tube 47 that is insertable into an esophagus (gullet) 34 (see FIG. 5) of a patient.

A balloon probe 46 is located at a first, proximal end 48a of the catheter tube 47.

A second, distal end 48b of the catheter tube 47 is connected to a measuring fluid port 90 of the device 60 for detecting the esophageal balloon pressure in such a manner that the catheter 48 may have applied thereto, in particular may be filled with, a fluid, in particular air, by the device 60. The device 60 for detecting the esophageal balloon pressure is also capable of removing fluid from the catheter 48 through the measuring fluid port 90 in order to empty the catheter 48, in particular the balloon probe 46 of the catheter 48.

The measuring fluid port 90 on the device 60 may be, for example, a male Luer Lock fitting or a tube connector that is compatible with a Luer Lock fitting, so that both plastic tubing and Luer connectors may be attached to the measuring fluid port 90.

The fluid connection between the second, distal end 48b of the catheter tube 47 and the device 60 for detecting the esophageal balloon pressure at the measuring fluid port 90 is detachable, so that the catheter 48 is selectively connectable to and disconnectable from the device 60 for detecting the esophageal balloon pressure.

The device 60 for detecting the esophageal balloon pressure comprises a fluid conveying device 65 adapted to selectively introduce fluid into the catheter 48 and to remove fluid from the catheter 48. The fluid conveying device 65 is in fluid communication with the catheter tube 47 at the measuring fluid port 90 and comprises, in particular, a pump device 100 and at least one valve 107.

The device 60 for detecting the esophageal balloon pressure further comprises a flow sensor 62, in particular a mass flow sensor 62, which is configured to determine the amount, i.e. the volume and/or mass flow, of fluid introduced into the catheter 48 and/or the amount of fluid removed from the catheter 48, and a pressure sensor 63, which is configured to determine the fluid pressure in the catheter 48.

The flow sensor 62, the pressure sensor 63 and the valve 107 may each be formed as a component part of the fluid conveying device 65 (as shown in FIG. 1), or as separate elements of the device 60 for detecting the esophageal balloon pressure, i.e. separate from the fluid conveying device 65.

The device 60 for detecting the esophageal balloon pressure also includes at least one control unit 80 configured to determine the esophageal balloon pressure. Such a method includes, in particular, suitably controlling the fluid conveying device 65 to fill the catheter 48 with fluid and/or remove fluid from the catheter 48 in targeted manner.

The at least one control unit 80 may be implemented as a stand-alone component “in hardware”. Two or more control units 80 may also be integrated in a common component or in a common group of components. The at least one control unit 80 may also be integrated into a ventilation device 10 (see FIG. 5).

The at least one control unit 80 may also be realized as a computer program product, i.e. by a corresponding software program which is executed on a processor, in particular a microprocessor or microcontroller. In this case, the software may be kept on a suitable local storage medium or a storage medium that can be retrieved via a network. The software contains instructions coded as a computer program which, when the software is loaded into a RAM memory of the processor and translated into machine language, causes the processor to execute the procedures described herein in more detail. Mixed forms between realization in hardware and realization in software are also possible.

The control unit 80 may be configured to allow a catheter 48 to be filled with a defined amount of fluid to operate the catheter 48 in vivo, i.e., in the esophagus 34 of a patient in order to measure the esophageal balloon pressure in the esophagus 34 of the patient and to determine the transpulmonary pressure in the chest of the patient therefrom.

The control unit 80 may also be adapted to operate the device 60 for detecting the esophageal balloon pressure as a characterization system which is adapted to characterize and possibly classify the catheter 48 ex vivo, i.e. before it is inserted into the esophagus 34 of a patient.

The device 60 also comprises a memory device 70 which is configured to store at least one detected variable, e.g. the balloon pressure and/or a characteristic property or parameter of the catheter 48 which has been determined by the device 60, and to output it as required via an output device 72, for example an electronic interface, a display device (“screen”) and/or a printer.

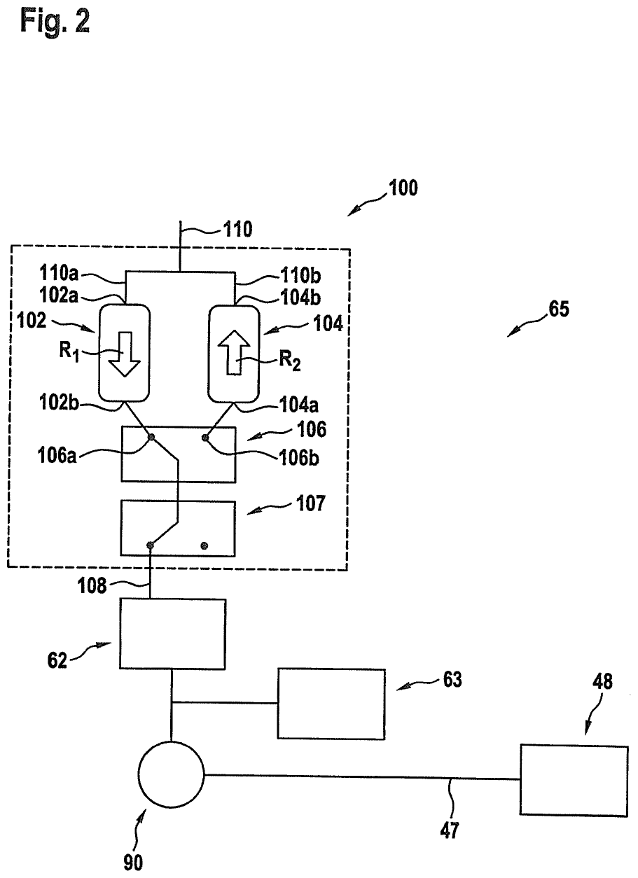

FIG. 2 shows a schematic view of a fluid conveying device 65 configured according to a first exemplary embodiment of the invention.

The fluid conveying device 65 comprises a pump device 100 having a first fluid pump 102 and a second fluid pump 104. The two fluid pumps 102, 104 in particular may be configured as micropumps 102, 104, for example as piezo pumps 102, 104.

Such micropumps/piezo pumps 102, 104 have a particularly compact design and are available at low cost.

The two fluid pumps 102, 104 are configured such that they each have a predetermined flow direction R₁ and R₂, respectively, in which the fluid is conveyed by the respective fluid pump 102, 104.

Each fluid pump 102, 104 therefore has a fluid inlet 102a, 104a, through which the conveyed fluid flows into the respective fluid pump 102, 104 during operation, and a fluid outlet 102b, 104b, through which the fluid leaves the respective fluid pump 102, 104 during operation.

The fluid pumps 102, 104 are connected to a switching valve 106 such that their flow directions R₁ and R₂, are opposite to each other. That is, the fluid outlet 102b of the first fluid pump 102 and the fluid inlet 104a of the second fluid pump 104 are connected to the switching valve 106.

The fluid inlet 102a of the first fluid pump 102 is in fluid communication with the environment via a feed line 110a, and the fluid outlet 104b of the second fluid pump 104 is in fluid communication with the environment via an drain line 110b. Thus, the fluid pumps 102, 104 can draw in air as fluid from the environment through the feed line 110a and discharge fluid into the environment through the drain line 110b.

The feed line 110a and the drain line 110b may be optionally combined to form a common feed and drain line 110.

If a fluid other than air is to be used as fluid, the feed line 110a may be fluidly connected to a corresponding fluid source, not shown in the figures, which provides the desired fluid, e.g. oxygen-enriched air.

The side of the switching valve 106 facing away from the fluid pumps 102, 104, shown below in FIG. 2, is fluidly connected to a fluid conveying line 108. By switching the switching valve 106, either the first fluid pump 102 or the second fluid pump 104 can be fluidly connected to the fluid conveying line 108.

A blocking valve 107 can optionally be provided in the fluid conveying line 108, which is configured to block any fluid flow through the fluid conveying line 108 in a blocking position and to enable a fluid flow through the fluid conveying line 108 in an enabling position.

The switching valve 106 may also be configured to additionally have a blocking position in which neither the first fluid pump 102 nor the second fluid pump 104 is connected to the fluid conveying line 108, and any fluid flow through the fluid conveying line 108 is blocked by the switching valve 106. In this case, a separate blocking valve 107 can be dispensed with.

A flow sensor 62 and a pressure sensor 63 are provided on the side of the blocking valve 107 facing away from the switching valve 106. The flow sensor 62 is configured to measure the fluid flow through the fluid conveying line 108, and the pressure sensor 63 is configured to measure the fluid pressure in the fluid conveying line 108.

On the side of the sensors 62, 63 facing away from the valves 106, 107 is the measuring fluid port 90, which allows the catheter tube 47 of the catheter 48 to be fluidly connected to the fluid conveying line 108.

When the catheter tube 47 of a catheter 48 is fluidly connected to the measuring fluid port 90, the blocking valve 107 is opened and the switching valve 106 is in a first position as shown in FIG. 2, fluid from the first fluid pump 102 can be conveyed from the feed line 110a into the catheter 48.

When the switching valve 106 is in a second position, not shown in FIG. 2, fluid may be withdrawn from the catheter 48 and discharged into the drain line 110b by operation of the second fluid pump 104.

In particular, the switching valve 106 may include a first release valve 106a associated with the first fluid pump 102 and a second release valve 106b associated with the second fluid pump 104.

In such a configuration, the first release valve 106a may be switchable at least between an open position, in which the fluid outlet 102b of the first fluid pump 102 is fluidly connected to the fluid conveying line 108, and a closed position, in which the fluid outlet 102b of the first fluid pump 102 is fluidly disconnected from the fluid conveying line 108.

Similarly, the second release valve 106b may be switchable at least between an open position, in which the fluid inlet 104a of the second fluid pump 104 is fluidly connected to the fluid conveying line 108, and a closed position, in which the fluid inlet 104a of the second fluid pump 104 is fluidly disconnected from the fluid conveying line 108.

In such a configuration, in which the switching valve 106 is formed with two release valves 106a, 106b, the previously described function of the switching valve 106, to switch the conveying direction of the fluid conveying device 65, can be realized by opening and closing the two release valves 106a, 106b in selective or targeted manner.

In particular, the two release valves 106a, 106b can be arranged in parallel to each other so that opening or releasing one of the two release valves 106a, 106b is sufficient to allow fluid to flow through the switching valve 106.

The switching valve 106 and in particular the two release valves 106a, 106b can be configured as mechanically, electrically, hydraulically and/or pneumatically controllable valves.

The first and second release valves 106a, 106b can be mechanically, hydraulically, pneumatically and/or electrically coordinated or synchronized with each other such that when the first release valve 106a is in its open position, the second release valve 106b is in its closed position, and that when the first release valve 106a is in its closed position, the second release valve 106b is in its open position.

In this way, it can be ensured that the switching valve can (only) be switched between two well-defined operating states, in each of which a respective one of the release valves 106a, 106b is open and the other one of the release valves 106a, 106b is closed.

The pump device 100 can also be configured such that the first fluid pump 102 can pump against a closed blocking valve 107 to adjust the system pressure at the fluid outlet 102b to the pressure in the esophageal balloon catheter 48, instead of pumping fluid into the environment to bring the first fluid pump 102 to the desired conveying capacity. In this case, the pressure sensor 63 or an additional pressure sensor, which is not shown in FIG. 2, would have to be provided between the fluid pump 102 and the blocking valve 107 in order to be able to measure the fluid pressure at the fluid outlet 102b of the first fluid pump 102 and to set it to the desired value.

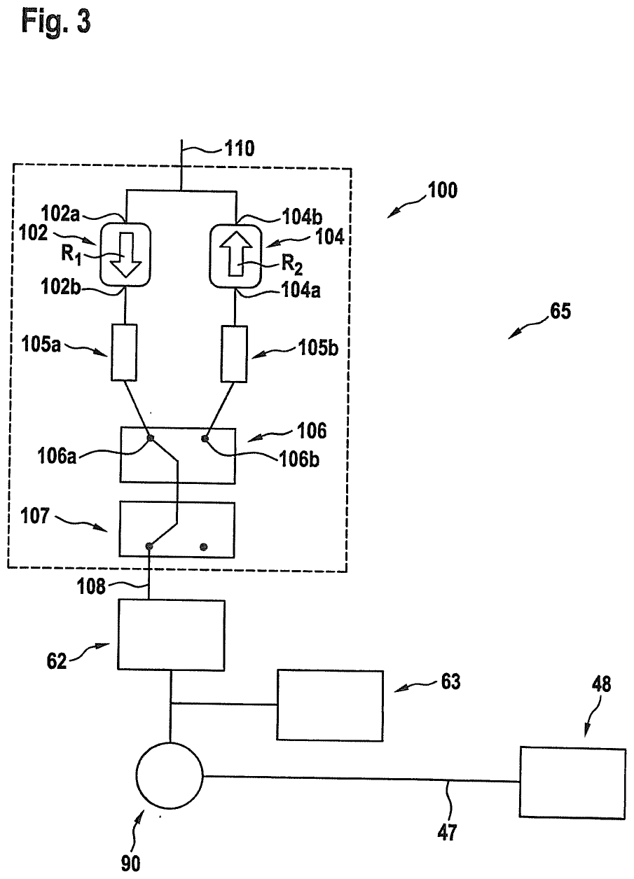

FIG. 3 shows a schematic view of a fluid conveying device 65 configured according to a second exemplary embodiment of the invention.

The structure of the fluid conveying device 65 shown in FIG. 3, which is configured according to the second exemplary embodiment of the invention, substantially corresponds to the structure of the fluid conveying device 65 configured according to the first exemplary embodiment shown in FIG. 2. The features of the second exemplar embodiment, which correspond to features of the first exemplary embodiment, are designated with the same reference numerals and will not be described again in detail in order to avoid repetitions. Instead, reference is made to the description of the first exemplary embodiment.

The fluid conveying device 65 according to the second exemplary embodiment differs from the fluid conveying device 65 according to the first exemplary embodiment in that, in the fluid conveying device 65 according to the second exemplary embodiment, a device 105a, 105b for forming a flow resistance is provided between the first fluid pump 102 and the switching valve 106 and between the second fluid pump 104 and the switching valve 106, respectively, which device is configured to reduce or limit a measuring fluid flow to a predetermined value at maximum pumping capacity of the first fluid pump 102 and/or the second fluid pump 104.

A device 105a, 105b for forming a flow resistance in particular may comprise a constriction of the flow cross-section, e.g. in the form of an orifice or throttle. Alternatively or additionally, the device 105a, 105b for forming a flow resistance may comprise a porous material, e.g. a foam, which is introduced into the flow path of the fluid in order to reduce or limit the measuring fluid flow.

The provision of such devices 105a, 105b for forming a flow resistance is particularly useful when the nominal conveying capacities of the fluid pumps 102, 104 are greater than required for the intended introduction and/or removal of fluid from the catheter 48 and there is a risk that the catheter 48 will be damaged by the fluid flow achievable at the maximum conveying capacity of the fluid pumps 102, 104 and/or that a desired process cannot be properly performed due to the high pressure and/or due to the high fluid flow.

The provision of a device 105a, 105b for forming a flow resistance also allows for a more accurate adjustment of the flow rate, since the fluid pump 102, 104 does not have to be operated in a range with a very low conveying capacity, but can be operated in a higher range in which the flow rate of the fluid pump 102, 104 can be better adjusted.

This can occur in particular if at least one of the fluid pumps 102, 104 is configured as a diaphragm pump, as diaphragm pumps generally have a higher rated conveying capacity than micropumps/piezo pumps.

In the exemplary embodiment shown in FIG. 3, the devices 105a, 105b for forming a flow resistance are arranged between the fluid pumps 102, 104, which are configured in particular as diaphragm pumps, and the switching valve 106. The devices 105a, 105b for forming a flow resistance are thus arranged downstream of the fluid outlet 102b of the first fluid pump 102 and upstream of the fluid inlet 104a of the second fluid pump 104.

In alternative exemplary embodiments not explicitly shown in the figures, at least one of the devices 105a, 105b for forming a flow resistance may also be arranged on the “other side” of the respective fluid pump 102, 104, i.e. upstream of the respective fluid pump 102, 104, at the fluid inlet 102a of the first fluid pump 102, or downstream, at the fluid outlet 104b of the second fluid pump 104.

In a further exemplary embodiment not explicitly shown in the figures, a device 105a, 105b common to both fluid pumps 102, 104 may also be provided for forming a flow resistance, through which fluid flows in one or the other direction depending on the position of the switching valve 106.

Such a common device 105a, 105b for forming a flow resistance may be arranged, for example, in a common feed and drain line 110 and/or on the side of the switching valve 106 facing away from the fluid pumps 102, 104 in the fluid conveying line 108, for example between the switching valve 106 and the blocking valve 107 or between the blocking valve 107 and the flow sensor 62.

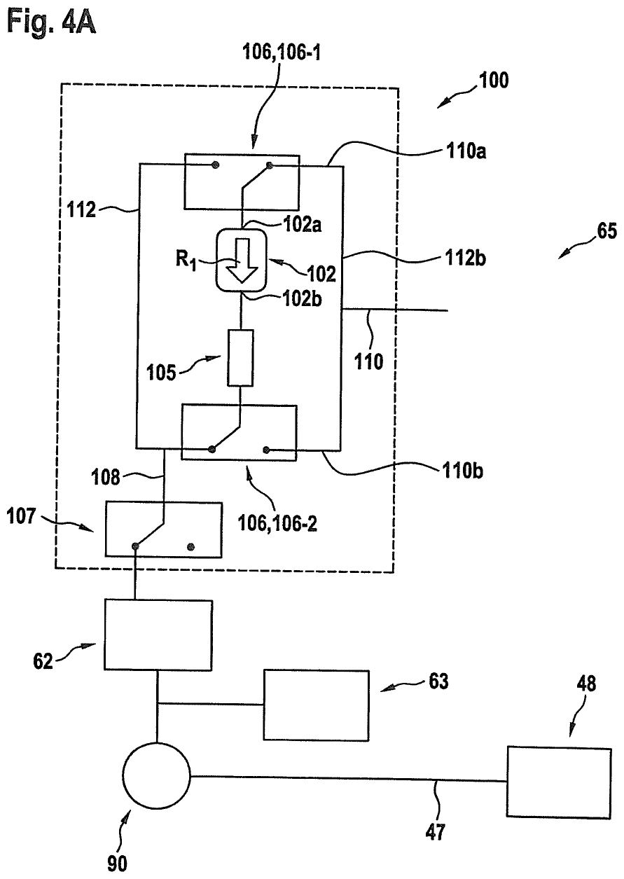

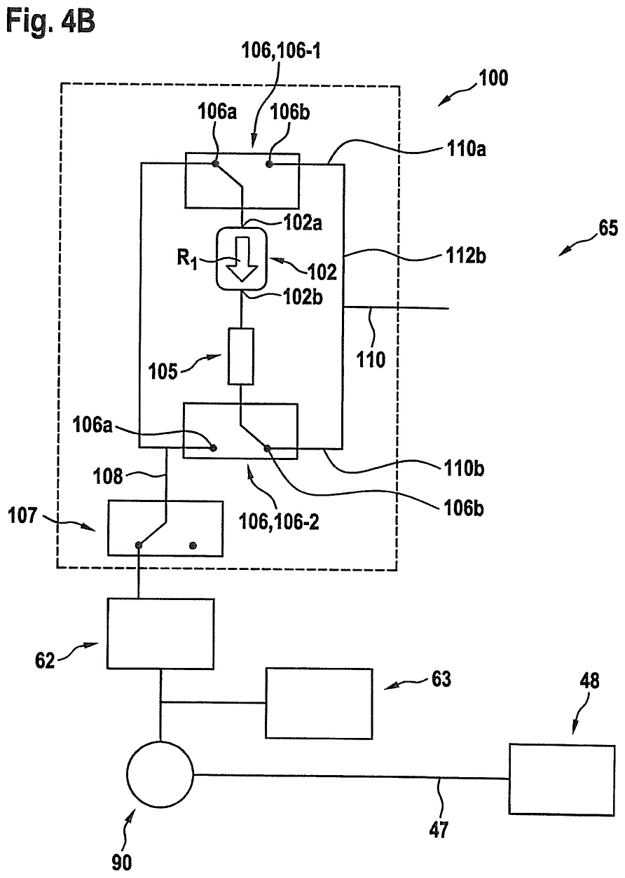

FIGS. 4A and 4B show schematic views of a fluid conveying device 65 configured according to a third exemplary embodiment of the invention. FIGS. 4A and 4B show the fluid conveying device 65 in two different operating states.

The pump device 100 of a fluid conveying device 65 configured according to a third exemplary embodiment of the invention comprises only a single fluid pump 102, which is configured to convey fluid in a predetermined flow direction R₁ from a fluid inlet 102a to a fluid outlet 102b of the fluid pump 102.

In a fluid conveying device 65 configured according to a third exemplary embodiment of the invention, the switching valve 106 comprises a first conveying direction switching valve 106-1 fluidly connected to the fluid inlet 102a of the fluid pump 102 so that it is arranged upstream of the fluid pump 102, and a second conveying direction switching valve 106-2 fluidly connected to the fluid outlet 102b of the fluid pump 102 so that it is arranged downstream of the fluid pump 102.

The first conveying direction switching valve 106-1 and the second conveying direction switching valve 106-2 thus are arranged in series with each other with respect to the flow direction R₁ of the measuring fluid, so that the two conveying direction switching valves 106-1, 106-2 are arranged one behind the other in the flow direction R₁ of the measuring fluid, with the fluid pump 102 being arranged between the two conveying direction switching valves 106-1, 106-2. Such a series arrangement of the two conveying direction switching valves 106-1, 106-2 has the consequence that in a hypothetical case in which at least one of the two conveying direction switching valves 106-1, 106-2 blocks the fluid flow, no fluid could be conveyed through the fluid pump 102.

The two conveying direction switching valves 106-1, 106-2 are each switchable between at least a first and a second open position.

The two conveying direction switching valves 106-1, 106-2 may each comprise a first release valve 106a and second release valve 106b, as has been described in connection with the first exemplary embodiment.

In FIG. 4A, the fluid conveying device 65 is shown in an operating state in which the two conveying direction switching valves 106-1, 106-2 are in the first open position.

In the first open position of the first conveying direction switching valve 106-1, the fluid inlet 102a of the fluid pump 102 is fluidly connected to the feed line 110a by the first conveying direction switching valve 106-1. In the first open position of the second conveying direction switching valve 106-2, the fluid outlet 102b of the fluid pump 102 is fluidly connected to the fluid conveying line 108 by the second conveying direction switching valve 106-2.

FIG. 4B shows a state in which both conveying direction switching valves 106-1, 106-2 are in the second open position.

In the second open position of the first conveying direction switching valve 106a, the fluid inlet 102a of the fluid pump 102 is fluidly connected to the fluid conveying line 108 by the first conveying direction switching valve 106-1. In the second open position of the second conveying direction switching valve 106-2, the fluid outlet 102b of the fluid pump 102 is fluidly connected to the drain line 110b by the second conveying direction switching valve 106-2.

The first conveying direction switching valve 106-1 and the second conveying direction switching valve 106-2 may be mechanically, hydraulically, pneumatically and/or electrically controllable valves.

The first conveying direction switching valve 106-1 and the second conveying direction switching valve 106-2 in particular may be mechanically, hydraulically, pneumatically and/or electrically coordinated or synchronized with each other such that, when the first conveying direction switching valve 106-1 is in the first open position, the second conveying direction switching valve 106-2 is also in the first open position, as shown in FIG. 4A, and that, when the first conveying direction switching valve 106-1 is in the second open position, the second conveying direction switching valve 106-2 is also in the second open position, as shown in FIG. 4B.

When the first conveying direction switching valve 106-1 and the second conveying direction switching valve 106-2 are in the first open position, as shown in FIG. 4A, fluid is conveyed from the feed line 110a into the fluid conveying line 108 during operation of the fluid pump 102.

The structure of the fluid conveying device 65 according to the third exemplary embodiment shown in the lower portion of FIGS. 4A and 4B, which comprises the blocking valve 107, the flow sensor 62, the pressure sensor 63, and the measuring fluid port 90, corresponds to the structure of the fluid conveying devices 65 according to the first exemplary embodiment and the second exemplary embodiment as shown in FIGS. 2 and 3 and described in this context. Therefore, this part of the fluid conveying device 65 will not be described again. Instead, reference is made to the description of FIGS. 2 and 3.

When the first conveying direction switching valve 106-1 and the second conveying direction switching valve 106-2 are in their respective second open position, as shown in FIG. 4B, fluid is conveyed during operation of the fluid pump 102 from the catheter 48 through the catheter tube 47 and the fluid conveying line 108, out of the catheter 48 into the drain line 110b, so that the catheter 48 is emptied.

Therefore, with a fluid conveying device 65 according to the third exemplary embodiment, as shown in FIGS. 4A and 4B, the catheter 48 can be both filled with fluid and emptied by a single fluid pump 102.

When the first conveying direction switching valve 106-1 and the second conveying direction switching valve 106-2 are not fixedly coupled and synchronized with each other, so that the synchronization between the two conveying direction switching valves 106-1, 106-2 may be canceled, an operating state of the fluid conveying device 65 may be set in which the first conveying direction switching valve 106-1 is in the first open position, as shown in FIG. 4A, and the second conveying direction switching valve 106-2 is in the second open position, as shown in FIG. 4B.

Such an operating state may be set to bring the conveying capacity of the fluid pump 102 to its maximum achievable value during operation, before the second conveying direction switching valve 106-2 is switched to the first open position to convey fluid into the catheter 48. In this way, the fluid can be conveyed into the catheter 48 at a defined conveying capacity from the beginning.

It is also possible to set an operating state in which the first conveying direction switching valve 106-1 is in the second open position (see FIG. 4B), and the second conveying direction switching valve 106-2 is in the first open position (see FIG. 4A). In such an operating state, fluid is conveyed by the fluid pump 102 in a circuit. This operating state may be used to flush the fluid pump 102 and the conveying direction switching valves 106-1, 106-2 with fluid.

Also in the third exemplary embodiment, a device 105 for forming a flow resistance may be provided at the fluid outlet 102b of the fluid pump 102, which is configured to reduce or limit the measuring fluid flow achievable at maximum pumping capacity of the fluid pump 102 to a predetermined value in order to avoid damage to the catheter 48 due to an excessive fluid flow.

The provision of a device 105a, 105b for forming a flow resistance also permits a more accurate adjustment of the flow rate, since the fluid pump 102, 104 does not have to be operated in a range with a very low conveying capacity, but may be operated in a higher range in which the flow rate of the fluid pump 102, 104 can be better adjusted.

As has been described in connection with the second exemplary embodiment (see FIG. 3), the device 105 for forming a flow resistance may also be arranged at other locations along the fluid flow, in particular upstream of the fluid inlet 102a of the fluid pump 102 and/or downstream of the second conveying direction switching valve 106-2 in the fluid conveying line 108.

When a fluid pump 102 with a low maximum conveying capacity, in particular a micropump, e.g. a piezo pump, is used as the fluid pump 102 so that there is no risk of damaging the catheter 48 by an excessive fluid flow, a device 105 for forming a flow resistance can be dispensed with in the third exemplary embodiment as well.

As described in connection with the first exemplary embodiment, a separate blocking valve 107 can be dispensed with if the switching valve 106, in this case the two conveying direction switching valves 106-1, 106-2 which together form the switching valve 106, has a blocking position in which fluid flow through the fluid conveying line 108 is blocked by the switching valve 106.

FIG. 5 shows, in a highly schematic representation and partly in the form of a block diagram, the essential elements of a ventilation device 10 comprising a fluid conveying device 65 according to the invention.

The ventilation device 10 is shown in FIG. 5 in a state with intubated windpipe trachea 12 of a ventilated patient. In addition to the trachea 12, the lung lobes 28, 30, the heart 32, the esophagus 34 and the thoracic wall 42 of the patient are shown very schematically in FIG. 5. The tube 14 of the ventilation device 10 is inserted, generally through the patient's mouth opening which is not shown, a short distance into the trachea 12 to supply breathing gas to the airway. Exhaled air is also discharged through tube 14, which branches at its upstream end into a first end 16 and a second end 22. The first end 16 is connected via an airway inlet valve 18 to an airway inlet port of the ventilation device 10 for applying an inspiration pressure PInsp. In the open position of the airway inlet valve 18, the inspiration pressure PInsp is applied to the airway. The second end 22 is connected via an airway outlet valve 24 to an airway outlet port of the ventilation device 10 for application of an expiration pressure PExp. In the open position of the airway outlet valve 24, the expiration pressure PExp is applied to the airway.

Both the inspiration pressure PInsp and the expiration pressure PExp are generated by the ventilation device 10 according to predetermined time patterns, such that inspiratory breathing gas flows toward the patient's lungs 28, 30 during an inspiration phase, as indicated by arrow 20 in FIG. 5, and expiratory breathing gas flows back from the patient's lungs 28, 30 during an expiration phase, as indicated by arrow 26. Normally, the airway inlet valve 18 remains open during the inspiration phase, and the inspiration pressure PInsp—which is usually greater than the expiration pressure PExp—is applied to the airway inlet. During the expiration phase, the airway inlet valve 18 is closed and the airway outlet valve 24 is open. Then, the expiration pressure PExp is applied to the airway inlet.

There can be used any forms of known ventilation modes, for example, pressure-controlled ventilation modes, volume-controlled ventilation modes, or ventilation modes in which pressure-controlled and volume-controlled aspects are combined. In addition to purely machine-controlled forms of ventilation, in which the time course of the inspiration pressure PInsp and possibly also of the expiration pressure PExp are determined by the ventilation device 10, it is also conceivable to have forms of ventilation in which the patient's spontaneous breathing efforts can either support the machine ventilation or the machine ventilation serves to support the patient's spontaneous breathing efforts. In such forms of ventilation, the time course of inspiration pressure PInsp or expiration pressure PExp and frequently also the position of inlet valve 18 or outlet valve 24 are not determined solely by the ventilation device 10, but are influenced by the patient's spontaneous breathing efforts.

The calibration of an esophageal catheter with balloon probe, which is introducable into the esophagus, for detecting an esophageal pressure Peso by means of which the transpulmonary pressure Ptp can be inferred, is particularly tailored to forms of ventilation in which ventilation is carried out by means of fully automatic ventilation modes, for example, ventilation by means of closed control loops, such as those used in Adaptive Support Ventilation (ASV ventilation) developed by the applicant and in INTELLIVENT ASV ventilation developed by the applicant as well. Such forms of ventilation are characterized by the fact that only minimal manual intervention by the operator is required and that the ventilation device automatically sets or adjusts important ventilation parameters such as the positive end-expiratory pressure PEEP or the maximum airway pressure Paw_max within predefined value ranges using suitable closed control loops.

The breathing gas may contain ambient air, but will typically contain a predetermined proportion of pure oxygen, hereafter referred to as FiO2, which is above the oxygen content of ambient air. The breathing gas will also typically be humidified.

The flow of the breathing gas at the airway entrance or inlet is determined using an airway inlet flow sensor 36. The airway inlet flow sensor 36 is based on detecting a pressure difference dP between an input volume 38 and an output volume 40 in communication with the input volume 38, and provides a determination of the breathing gas mass flow at the airway inlet. At the same time, the value of the airway inlet pressure Paw can be derived quite easily from the pressure signal in the output volume 40.

The pressure prevailing in the alveoli of the lungs 28, 30 is indicated by Palv in FIG. 5. This pressure depends on the airway inlet pressure Paw as well as the flow of the breathing gas V into or out of the lungs and the airway resistance R. In the case of pressure equalization between the airway inlet and the alveoli, the alveolar pressure Palv is equal to the airway inlet pressure. Such a pressure equalization has the consequence that the breathing gas flow V′ comes to a standstill. For example, a brief occlusion maneuver of the airway, i.e. airway inlet valve 18 and airway outlet valve 24 remain closed at the same time, can lead to pressure equalization. In this case, the occlusion maneuver must last just long enough for the gas flow V in the airway to stop. This is usually between 1 and 5 s. In this state, the alveolar pressure Palv can then be determined by determination of the airway inlet pressure Paw.

Both in physiological breathing and in mechanical ventilation, the flow of breathing gas is determined by a pressure difference between the alveolar pressure Palv and the airway inlet pressure Paw.

In the case of purely physiological breathing, a negative pressure difference, i.e. a negative pressure, between the alveolar pressure Palv and the airway inlet pressure Paw is generated for inhalation by expansion of the thorax (indicated at 42 in FIG. 5) and associated lowering of the pressure Ppl in the pleural space or gap 44 formed between the thorax 42 and the lungs 28, 30. Exhalation takes place passively by relaxation of the thorax and elastic resetting of the lung tissue. For this reason, during physiological breathing, the pressure in the pleural gap Ppl is always lower than the alveolar pressure Palv. The transpulmonary pressure Ptp, defined as the difference between the alveolar pressure Palv and the pressure in the pleural gap Ppl, is thus generally positive and becomes zero in the case of complete pressure equalization.

During mechanical ventilation, the breathing gas is pumped into the lungs at a positive pressure. For this reason, in mechanical ventilation, the airway inlet pressure Paw=PInsp is greater than the alveolar pressure Palv and the latter in turn is greater than the pressure in the pleural gap Ppl during the inspiration phase. It follows from these pressure relationships that the transpulmonary pressure Ppl in mechanical ventilation is positive during inspiration. During expiration, the airway inlet has an airway pressure Pexp applied thereto that is lower than the alveolar pressure Palv, so that breathing gas flows out of the alveoli. In the case of a very low airway pressure PExp, it may happen at the end of expiration, when very little gas is left in the lungs, that the pressure in the pleural gap Ppl exceeds the alveolar pressure Palv to such a high extent that part of the alveoli of the lungs collapse. The transpulmonary pressure Ptp is then negative.

Collapsing of the alveoli may be prevented by applying an additional positive pressure to the airway inlet also during the expiration phase. A positive airway pressure is then permanently applied to the airway inlet, i.e. during the inspiration phase and also during the expiration phase. This positive airway pressure is referred to as positive end-expiratory pressure or PEEP.

Consequently, the transpulmonary pressure Ptp is a suitable parameter for setting the PEEP. However, the transpulmonary pressure Ptp is not amenable to direct detection and cannot be determined from the pressures regularly detected during mechanical ventilation, as described above, either.

In FIG. 5, a balloon probe 46 for measuring the pressure in the esophagus 34, referred to as esophageal pressure Pₑₛₒ, is indicated in schematic manner. The balloon probe 46 is in the form of a balloon and is attached to the catheter 48 inserted into the esophagus 34. When the catheter 48 is inserted into an esophagus 34, the balloon probe 46 abuts internally against the wall of the esophagus 34 and provides the pressure applied to the esophagus 34 at the location of the balloon probe 46. This pressure is a good approximation of the pressure Ppl in the pleural gap when the patient is positioned appropriately. The described balloon probe 46 for detecting the esophageal pressure Pₑₛₒ and the handling of this probe is described, for example, in Benditt J., Resp. Care, 2005, 50: pp. 68-77.

The ventilation device 10 comprises furthermore a device 60 for detecting an esophageal balloon pressure, as shown in FIG. 1. The device 60 may be configured to provide the function of a characterization system 60 that allows the catheter 48 to be characterized and possibly classified ex vivo, i.e., before it is inserted into the patient's esophagus 34.

1. A fluid conveying device for introducing fluid into and/or removing fluid from an esophageal catheter which is insertable into an esophagus and which has a balloon probe for determining an esophageal balloon pressure, comprising:
a measuring fluid port which is configured to be coupled to a distal end of the esophageal catheter such that, in a coupled state, fluid is introducible into the esophageal catheter and/or removable from the esophageal catheter via the measuring fluid port;
a pump device having at least one fluid pump, wherein the pump device is configured to convey fluid in at least one conveying direction; and
a switching valve which is associated with the fluid pump and which is switchable at least between a first position and a second position;
wherein the fluid pump is provided in the form of a micropump or diaphragm pump.

2. The fluid conveying device according to claim 1,
wherein the fluid pump is a first fluid pump for conveying fluid in a first flow direction and wherein the pump device comprises a second fluid pump for conveying fluid in a second flow direction, wherein the second fluid pump in particular is in the form of a micropump or diaphragm pump.

3. The fluid conveying device according to claim 2,
wherein the first fluid pump and the second fluid pump are connected in parallel to each other; and/or
wherein the switching valve is configured such that it selectively connects the measuring fluid port either to the first fluid pump or to the second fluid pump; and/or
wherein the switching valve is arranged between the outlet of the first fluid pump and the measuring fluid port and/or between the inlet of the second fluid pump and the measuring fluid port.

4-5. (canceled)

6. The fluid conveying device according to claim 3,
wherein the switching valve is switchable at least between a first and a second open position;
wherein, in the first open position, an outlet of the first fluid pump is fluidly connected to the measuring fluid port and an inlet of the second fluid pump is fluidly disconnected from the measuring fluid port; and
wherein, in the second open position, an inlet of the second fluid pump is fluidly connected to the measuring fluid port and an outlet of the first fluid pump is fluidly disconnected from the measuring fluid port.

7. The fluid conveying device according to claim 6,
wherein the switching valve comprises a first release valve associated with the first fluid pump and a second release valve associated with the second fluid pump;
wherein the first release valve is switchable at least between an open position, in which the outlet of the first fluid pump is fluidly connected to the measuring fluid port, and a closed position, in which the outlet of the first fluid pump is fluidly disconnected from the measuring fluid port; and
wherein the second release valve is switchable at least between an open position, in which the inlet of the second fluid pump is fluidly connected to the measuring fluid port, and a closed position, in which the inlet of the second fluid pump is fluidly disconnected from the measuring fluid port.

8. The fluid conveying device according to claim 7,
wherein the first release valve and the second release valve are arranged in parallel to each other; and/or
wherein the first release valve and the second release valve are switchable in coordinated or synchronized manner with each other such that when the first release valve is in the open position, the second release valve is in the closed position, and when the first release valve is in the closed position, the second release valve is in the open position.

9. (canceled)

10. The fluid conveying device according to claim 1,
wherein the switching valve is configured such that it selectively connects the measuring fluid port either to an inlet of the first fluid pump or to an outlet of the fluid pump.

11. The fluid conveying device according to claim 10,
wherein the switching valve comprises a first conveying direction switching valve and a second conveying direction switching valve which, with respect to the direction of flow of fluid through the fluid pump, are arranged in series with each other.

12. The fluid conveying device according to claim 11,
wherein the first conveying direction switching valve is located downstream of the first fluid pump with respect to the direction of flow of fluid through the fluid pump; and
wherein the second conveying direction switching valve is located upstream of the fluid pump with respect to the direction of flow of fluid through the fluid pump; and/or
wherein the first conveying direction switching valve is switchable between at least a first and a second open position;
wherein, in the first open position, the outlet of the fluid pump is fluidly connected to the measuring fluid port; and
wherein in the second open position, the outlet of the fluid pump is fluidly disconnected from the measuring fluid port.
wherein the second conveying direction switching valve is switchable between at least a first and a second open position;
wherein in the first open position, the inlet of the fluid pump is fluidly disconnected from the measuring fluid port; and
wherein in the second open position, the inlet of the fluid pump is fluidly connected to the measuring fluid port; and/or
wherein the first conveying direction switching valve and the second conveying direction switching valve are switchable in coordinated or synchronized manner with each other such that when the first conveying direction switching valve is in the first open position, the second conveying direction switching valve is also in the first open position; and/or
wherein the first conveying direction switching valve and the second conveying direction switching valve are switchable in coordinated or synchronized manner with each other such that when the first conveying direction switching valve is in the second open position, the second conveying direction switching valve is also in the second open position.

13-16. (canceled)

17. The fluid conveying device according to claim 1,
comprising a blocking valve comprising at least one blocking position in which the pump device is fluidly disconnected from the measuring fluid port.

18. The fluid conveying device according to claim 17,
wherein the blocking valve is configured as a valve 1which is separate from the switching valve and which is arranged in series with the switching valve.

19. The fluid conveying device according to claim 1,
wherein the switching valve comprises a blocking position in which the pump device is fluidly disconnected from the measuring fluid port.

20. The fluid conveying device 65 according to claim 1,
wherein the first micropump is designed as a piezo pump and/or wherein the second micropump is designed as a piezo pump

21. The fluid conveying device according to claim 1,
wherein the first fluid pump and/or the second fluid pump each have an associated device for forming a flow resistance, which is configured such that it reduces a measuring fluid flow which is achievable at maximum pumping capacity of the first fluid pump and/or the second fluid pump, to a predetermined value due to the flow resistance.

22. The fluid conveying device according to claim 1,
further comprising a flow sensor configured to detect a measuring fluid flow conveyed by the first fluid pump and/or by the second fluid pump,

23. The fluid conveying device according to claim 21, wherein the flow sensor is configured to detect a mass flow of the fluid or volume flow of the fluid conveyed by the first fluid pump and/or by the second fluid pump.

24. The fluid conveying device according to claim 21, wherein the flow sensor is arranged between the measuring fluid port and the first fluid pump and/or between the measuring fluid port and the second fluid pump.

25. The fluid conveying device according to claim 1,
further comprising a pressure sensor configured to measure a fluid pressure of the measuring fluid conveyed by the first fluid pump and/or by the second fluid pump in a region between the measuring fluid port and the first fluid pump and/or between the measuring fluid port and the second fluid pump.

26. A device for detecting an esophageal balloon pressure on the basis of an esophageal catheter with balloon probe, which is insertable into an esophagus, the device comprising a fluid conveying device according claim 1.

27. A ventilation device comprising a device for detecting an esophageal balloon pressure according to claim 26.
